(19) United States
(12) Patent Application Publication
(10) Pub. No.: US 2006/0247638 A1
(43) Pub. Date: Nov. 2, 2006

(54) Composite spinal fixation systems

(75) Inventors: Hai H. Trieu, Cordova, TN (US); Fred Molz, Collierville, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/117,516

(22) Filed: Apr. 29, 2005

(51) Int. Cl. A61F 2/30

(52) U.S. Cl. 606/69

(57) ABSTRACT

Embodiments provide composite components for use in spinal fixation systems. The composite components may comprise polyetheretherketone (PEEK) or another non-resorbable or resorbable polymeric material and at least one metal. Incorporation of PEEK or another non-resorbable or resorbable polymeric material into the components allows average or mean physical properties (e.g., tensile strength, modulus of elasticity, etc.) of the components to be modulated. The composition and orientation of the composite components can be advantageously chosen to produce components with desired physical characteristics.

### FIELD OF THE INVENTION

Embodiments of the invention relate to spinal fixation systems having at least one composite component. More particularly, the embodiments relate to rods and plates for use in spinal fixation systems that are composites of polyetheretherketone (PEEK) and metals or metal alloys.

### BACKGROUND

The spinal (vertebral) column is a biomechanical structure composed primarily of ligaments, muscles, vertebrae, and intervertebral discs. The biomechanical functions of the spinal column include (i) support of the body; (ii) regulation of motion between the head, trunk, arms, pelvis, and legs; and (iii) protection of the spinal cord and the nerve roots. Damage to one or more components of the spinal column, such as an intervertebral disc, may result from disease or trauma and cause instability of the spinal column. To prevent further damage and overcome some of the symptoms resulting from a damaged spinal column, a spinal fixation device may be installed to stabilize the spinal column.

A spinal fixation device generally consists of stabilizing elements, such as rods or plates, attached by anchors to the vertebrae in the section of the vertebral column that is to be stabilized. The spinal fixation device restricts the movement of the fixed vertebrae relative to one another and supports at least a part of the stresses that would otherwise be imparted to the vertebral column. Typically, the stabilizing element is rigid and inflexible and is used in conjunction with an intervertebral fusion device to promote fusion between adjacent vertebral bodies. There are some disadvantages associated with the use of rigid spinal fixation devices, including decreased mobility, stress shielding (i.e. too little stress on some bones, leading to a decrease in bone density), and stress localization (i.e. too much stress on some bones, leading to fracture and other damage).

In response, flexible spinal fixation devices have been employed. These devices are designed to support at least a portion of the stresses imparted to the vertebral column but also allow a degree of movement. In this way, flexible spinal fixation devices avoid some of the disadvantages of rigid spinal fixation devices.

The description herein of problems and disadvantages of known apparatuses, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

What is needed is a method to fabricate spinal fixation systems, and systems so fabricated that have adjustable stiffness or flexibility. What also is needed are spinal fixation systems with good flexibility and good strength. Also, spinal fixations systems that are exceptionally biocompatible are needed. Embodiments of the invention solve some or all of these needs, as well as additional needs.

Therefore, in accordance with an embodiment of the present invention, there is provided a spinal fixation system having at least one composite component, the composite comprising a first material comprising at least one metal or metal alloy, and a second material selected from the group consisting of resorbable and non-resorbable polymeric materials.

In accordance with a further embodiment of the present invention, there is provided a spinal fixation system having at least one component comprised of a composite of PEEK and at least one metal or metal alloy. In another embodiment, there is provided composite spinal fixation rods comprising PEEK and at least one metal or metal alloy. In accordance with another embodiment of the present invention, there is provided composite spinal fixation plates comprising PEEK and at least one metal or metal alloy.

In accordance with another embodiment, there is provided a method of making a composite component of a spinal fixation system. The method comprises selecting a suitable design for the component and forming the composite component from at least one metal or metal alloy and a polymeric material selected from the group consisting of resorbable and non-resorbable polymeric materials.

These and other features and advantages of the embodiments will be apparent from the description provide herein.

### BRIEF DESCRIPTION OF THE DRAWINGS

FIG. 1, embodiments A, B, and C, illustrates an exemplary composite spinal fixation rod according to embodiments of the invention.

FIG. 2, embodiments A and B, illustrates an exemplary composite spinal fixation plate according to embodiments of the invention.

FIG. 3, embodiments A and B, illustrates an exemplary composite spinal fixation plate according to embodiments of the invention.

FIG. 4, embodiments A and B, illustrates an exemplary composite spinal fixation plate according to embodiments of the invention.

FIG. 5, embodiments A and B, illustrates an exemplary composite spinal fixation rod according to embodiments of the invention.

### DETAILED DESCRIPTION OF THE EMBODIMENTS

The following description is intended to convey a thorough understanding of the various embodiments of the invention by providing a number of specific embodiments and details involving spinal fixation systems having at least one composite component. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

It is a feature of an embodiment of the present invention to provide composite components, such as rods and plates, for use in spinal fixation systems. The composite components may comprise a first material comprising at least one metal or metal alloy; and a second material selected from the group consisting of resorbable and non-resorbable polymeric materials. In a preferred embodiment, the composite comprises polyetheretherketone and a metal or metal alloy.

Polyetheretherketone (PEEK) is a polymer with repeating mer units of Formula 1: 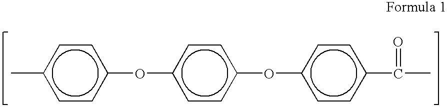
PEEK is commercially available from a number of suppliers and also is available in medical grades that are preferred for use in the embodiments (e.g., PEEK OPTIMA™, commercially available from Invibio Ltd., Lancashire, United Kingdom).

The resorbable and non-resorbable polymeric materials, such as PEEK, can be combined with at least one metal or metal alloy in accordance with the embodiments in order to form composite components such as rods and plates for use in spinal fixation systems. Preferred metal and metal alloys for use in the invention include, but are not limited to, titanium, titanium alloys (e.g. Ti-6Al-4V), tantalum, tantalum alloys, stainless steel alloys, cobalt-based alloys, cobalt-chromium alloys, cobalt-chromium-molybdenum alloys, niobium alloys, nickel-titanium alloys (Nitinol), and zirconium alloys.

The composite components of the embodiments include, but are not limited to, rods, plates, screws, clamps, and other components of spinal fixation systems. In preferred embodiments, there are provided composite rods and plates for spinal fixation systems. The composite spinal fixation rods and plates of the embodiments can be fabricated in any number of alternative forms. In one embodiment, a composite rod comprises a central rod of PEEK and an outer sheath of a metal. In another embodiment, a composite rod comprises a central rod of metal and an outer sheath of PEEK. In another embodiment, a composite plate comprises a central core of PEEK covered with an outer layer of a metal. In still another embodiment, a composite plate comprises a central core of metal covered with an outer layer of PEEK.

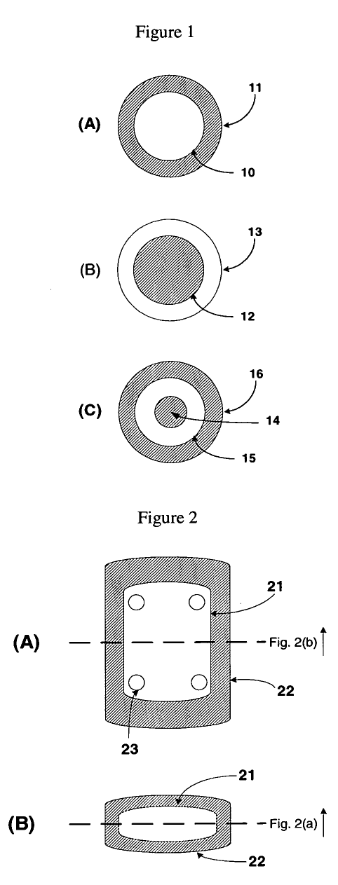

FIG. 1, embodiments A, B, and C, illustrates an exemplary composite rod for spinal fixation according to embodiments of the invention. Embodiment A illustrates a cross section of a spinal rod comprising a central rod or inner core of PEEK 10 and an outer sheath or covering of metal 11. Embodiment B illustrates a cross section of a spinal rod comprising a central rod or inner core of metal 12 and an outer sheath or covering of PEEK 13. In embodiment C, a more complex composite rod is depicted comprising a metallic core 14 and outer sheath 16 and an intermediary PEEK structure 15. FIG. 2, embodiments A and B, illustrates an exemplary composite plate for spinal fixation according to embodiments of the invention. Embodiment A illustrates a spinal fixation plate comprising a PEEK core 21 encased by a metal layer 22. Apertures 23 are provided for fixation of the composite plate to the vertebrae. Embodiment B shows a cross section of the composite plate.

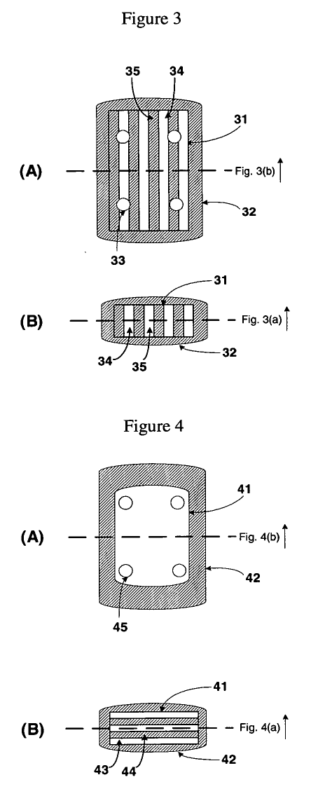

FIG. 3, embodiments A and B, illustrates another exemplary composite plate for spinal fixation. Embodiment A illustrates a core 31 of alternating PEEK 34 and metal strips 35 encased by a metal layer 32. Apertures 33 are provided for fixation of the composite plate to the vertebrae. Embodiment B shows a cross section of the composite plate.

FIG. 4, embodiments A and B, illustrates another exemplary composite plate for spinal fixation. Embodiment A illustrates a core 41 encased by a metal layer 42. An aperture 45 also is provided for fixation of the composite plate to the vertebrae. Embodiment B shows a cross section of the composite plate wherein the core 41 comprises a laminate of alternating PEEK 43 and metal 44 layers.

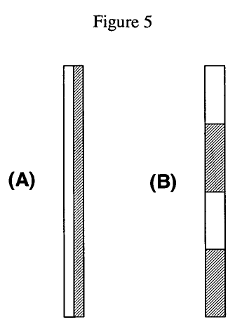

FIG. 5, embodiments A and B, illustrates an exemplary composite rod for spinal fixation according to embodiments of the invention. Embodiment A illustrates a longitudinal cross section of a spinal rod comprising a composite of PEEK and a metal. Embodiment B illustrates a longitudinal cross section of a spinal rod comprising alternating PEEK and metal portions.

It should be apparent that the composite components provided by the embodiments may take a myriad of different forms or configurations, in accordance with the guidelines provided herein. Therefore, one of skill in the art will appreciate still other configurations for composite spinal fixation components in accordance with the embodiments. For example, the metal and polymer portions of each composite component may have varying thicknesses and geometries, and need not correspond to the relatively uniform thicknesses and geometries depicted in the figures. Accordingly, skilled artisan will appreciate that an infinite number of variations in cross sections of the composite rods and plates provided for by the embodiments may occur, in accordance with the guidelines provided herein.

Although FIGS. 1-5 were illustrated with respect to PEEK/metal composites, according to embodiments of the invention other resorbable and non-resorbable polymeric materials may be used in place of PEEK in the composite structures. For example, a resorbable polymer material such as polylactides (PLA), polyglycolides (PGA), copolymers of (PLA and PGA), polyorthoesters, tyrosine, polycarbonates, and mixtures and combinations thereof may be used in lieu of PEEK. Also, non-resorbable polymeric material such as members of the polyaryletherketone family, polyurethanes, silicone polyurethanes, polyimides, polyetherimides, polysulfones, polyethersulfones, polyaramids, polyphenylene sulfides, and mixtures and combinations thereof alternatively may be used in lieu of PEEK. Therefore, a wide variety of composite components may be fabricated in accordance with the embodiments.

Described herein are some exemplary spinal fixation systems utilizing rods, plates, and other components. It is contemplated that the composite components of the present invention can be substituted for the rods, plates, and other components of these exemplary spinal fixation systems. Rods and plates and other spinal system components that are known for use with spinal fixation systems can be fabricated as composite rods or plates, as they are disclosed in embodiments of the present invention, using techniques known in the art and the guidelines provided herein. Additionally, the present invention contemplates that the composite rods, plates, and other components provided herein also may be utilized with future spinal fixation systems.

U.S. Pat. No. 6,858,029 discloses a system for fixing vertebrae comprising clamps and a connection portion to which the clamps may be mounted. The clamps are designed to engage vertebral bodies and the connection portion may comprise a rod. The system components disclosed in the '029 patent (e.g., rods, screws, etc.) may be fabricated using the composites described in the embodiments herein. The disclosure of U.S. Pat. No. 6,858,029 is incorporated herein by reference in its entirety.

U.S. Pat. No. 6,843,790 discloses a system for rigidly coupling at least three vertebrae. The system comprises an elongated plate having an upper and a lower surface, a first upper linear section, a second lower linear section, and a central curved section. The lower linear section and upper linear sections may be at an angle relative to each other. An opening is located within the central region of the plate and runs along the central axis of the plate. The plate may be affixed to the vertebrae by a plurality of bone engaging screws, each having a head for engaging the aperture in the plate. Components of the system disclosed in the '790 patent, including the elongated plate, may be fabricated using the composites described in the embodiments herein. The disclosure of U.S. Pat. No. 6,843,790 is incorporated herein by reference in its entirety.

U.S. Pat. No. 6,770,075 discloses a spinal fixation system including a plurality of anchor screw assemblies having anchor screws and clamp assemblies defining rod passages therethrough. A rod is receivable in the rod passages between the anchor screw assemblies, and a spacer is securable on the rod. Anchor screw assemblies can be affixed to adjacent vertebrae and the rod can be secured between the anchor screw assemblies, thereby fixing a relative spacing of the adjacent vertebrae. Components of the system disclosed in the '075 patent, including the fixation rods, may be fabricated using the composites described in the embodiments herein. The disclosure of U.S. Pat. No. 6,770,075 is incorporated herein by reference in its entirety.

U.S. Pat. No. 6,740,088 discloses a spinal fixation system comprising a plate having curvature in two planes such that it conforms to the curvature of the L5 vertebral body and to the patient's lordotic curve. The plate has holes for receiving screws to anchor the plate to the vertebral body and sacrum. The plate's base has a flange or foot portion to provide a wider base end area for support in the L5-S1 vertebral interspace. The foot portion also is arranged for appropriate entry angle of screws into the sacrum such as to improve anchorage in the sacrum. Components of the system disclosed in the '088 patent, including the curved plate, may be fabricated using the composites described in the embodiments herein. The disclosure of U.S. Pat. No. 6,740,088 is incorporated herein by reference in its entirety.

U.S. Pat. No. 6,706,044 discloses a spinal fixation system consisting of at least two bone anchors for attaching the device to the spine, at least two stacked rods running generally parallel to one another, means for connecting the rods to the bone anchors, and means for compressing the rods tightly together. The at least two stacked rods have a longitudinal shape and length, a cross sectional shape and cross sectional diameter, and are immediately adjacent one another along their length. Components of the system disclosed in the '044 patent, including the stacked rods, may be fabricated using the composites described in the embodiments herein. The disclosure of U.S. Pat. No. 6,706,044 is incorporated herein by reference in its entirety.

U.S. Pat. No. 6,613,051 discloses a spinal fixation system comprising a support member defining a plurality of engaging portions thereon. At least two of the engaging portions are spaced longitudinally from each other and are adapted to span at least one vertebra. At least two of the engaging portions are spaced laterally from each other and adapted to span a lateral distance of the vertebra. A plurality of fixation elements are provided to mount the engaging portions onto the vertebra. The support member thereby is restrained from rotational or translational movement relative to the vertebra. Components of the system disclosed in the '051 patent, including the engaging portions, may be fabricated using the composites described in the embodiments herein. The disclosure of U.S. Pat. No. 6,613,051 is incorporated herein by reference in its entirety.

U.S. Pat. No. 6,599,290 discloses a spinal fixation system comprising a plate member having multiple pairs of nodes. Each node defines a bone screw aperture. Linking segments connect the pairs of nodes to one another and elongated viewing windows are located between adjacent linking segments. Components of the system disclosed in the '290 patent, including the plate member, may be fabricated using the composites described in the embodiments herein. The disclosure of U.S. Pat. No. 6,599,290 is incorporated herein by reference in its entirety.

U.S. Pat. No. 6,547,790 discloses a bone plate that is T-shaped and includes two apertures, one on each arm of the T, to accommodate bolt anchor assemblies to which a linking member (e.g., a rod or cable) may be attached. Three chamfered holes extend along the midline of the bone plate for bone screws, and one additional bone screw opening is provided on each side arm of the bone plate to firmly fasten the plate. The arms of the plate may curve, or extend at a slight dihedral angle to the central line of the T to conform to the skull. Components of the system disclosed in the '790 patent, including the T-shaped bone plate, may be fabricated using the composites described in the embodiments herein. The disclosure of U.S. Pat. No. 6,547,790 is incorporated herein by reference in its entirety.

The spinal fixation systems, including rods and plates described herein, are exemplary only and it is to be understood that the composite systems, rods, and plates provided by embodiments of the invention can be fabricated to be physically similar in appearance and dimensions to any known system, rod, plate, or other component useful for spinal fixation. Therefore, the composite rods and plates of the present invention generally can act as substitutes for rods and plates of any given spinal fixation system. The composite rods and plates are not limited to a certain form or dimensions.

Table 1 compares some of the mechanical properties of PEEK and various metals and metal alloys. The composite rods and plates provided by the embodiments enable the production of devices and systems with custom properties.

TABLE 1
Properties of PEEK and Some Metals
and Metal Alloys (room temperature)

| | Modulus of | Tensile Strength |
|---|---|---|
| Material | Elasticity (GPa) | (MPa) |
| PEEK | 1.10 | 70.3-103 |
| Ti-6Al-4V | 114 | 900 |
| (annealed) | | |
| Ti (Cp) | 103 | 240 |
| (annealed) | | |
| Tantalum (Cp) | 185 | 205 |
| Stainless steel | 193 | 515 |
| 304 (hot finished | | |
| and annealed) | | |
| Stainless steel | 193 | 515 |
| 316 (hot finished | | |
| and annealed) | | |

Cp = commercially pure

As can be seen, PEEK generally has a lower modulus of elasticity and tensile strength than the exemplary metals and metal alloys shown in the table. The differences in physical properties between PEEK and the metals can be advantageously utilized in the embodiments by fabricating the composite spinal fixation systems, rods, plates, and other components with appropriate proportions of PEEK and metal, metal alloy, or mixtures thereof to produce a device having the desired physical properties. In this way, composite components can be fabricated having, for example, an average or mean modulus of elasticity different from that of the modulus of elasticity of any of its individual components. For example, consider two rods with the same diameter—the first rod of Ti-6Al-4V and the second rod a composite of Ti-6Al-4V and PEEK. Because a portion of the second rod comprises a material having a lower modulus of elasticity (PEEK), than the modulus of elasticity of Ti-6Al-4V, the second rod will have a lower average or mean modulus of elasticity than the first rod. In general, a composite rod will have average or mean properties, such as average or mean modulus of elasticity, proportionate to the ratio of the components that comprise the rod. One who is skilled in the art will appreciate how to select an appropriate ratio and orientation of the components that make up the systems, rods, plates, and other components based on the desired physical properties, in accordance with the guidelines described herein. For example, other polymeric materials such as those provided herein may be chosen for use in the composite components instead of PEEK, in order to produce composite components having different average or mean properties.

Fabricating composite components of spinal fixation systems may be advantageous because of the ability to produce composite components with average or mean properties not otherwise possible. For example, if a rod of a certain diameter is required for use with a given spinal fixation system, fabricating a composite rod having the required diameter using PEEK and metal composites may yield a composite rod with an average or mean modulus of elasticity not otherwise achievable for the required diameter rod, if fabricated from a non-composite composite material. Therefore, one advantage provided by the embodiments is that a spinal fixation system component may be fabricated having a different average or mean modulus of elasticity without changing the dimensions or geometry of the component. This may be highly advantageous, for example, where fixation systems are desired to be retrofitted or otherwise customized for use with patients that require a more flexible fixation system, but require components that imitate the dimensions and geometries of the original, non-composite components of the fixation systems. To aid these patients, composite components may be fabricated in accordance with embodiments herein.

In a preferred embodiment, composite spinal fixation rods and plates may be fabricated that have physical properties not otherwise attainable in rods and plates that are composed purely of metals and metal alloys. Preferably, the composite rods and plates have a mean or average modulus of elasticity less than about 75 GPa. Additionally, it is preferable that the composite rods and plates have a mean or average tensile strength less than about 150 MPa. One skilled in the art will be capable of fabricating composite materials comprising PEEK and at least one metal or metal alloy that have one or more of these preferred physical properties.

In another preferred embodiment, composite spinal fixation components may be fabricated comprising PEEK and a metal or metal alloy having a mean or average modulus of elasticity from about 1.2 GPa to about 192 GPa. More preferably, components may be fabricated having a mean or average modulus of elasticity from about 2 GPa to about 100 GPa. Even more preferably, components may be fabricated having a mean or average modulus of elasticity from about 3 GPa to about 50 GPa.

Fabricating the composite spinal fixation component preferably is carried out by utilizing a metal injection molding (MIM) technique to fabricate the metallic portion, and an injection molding technique to fabricate the non-metallic, or polymeric portion. For example, a MIM technique can be used to fabricate a composite spinal fixation component comprised of an outer metallic shell with an inner cavity. After molding, the inner cavity may be filled with polymer. Other techniques suitable for fabricating the composite spinal fixation components described herein also can be used, as will be appreciated by those skilled in the art upon review of the guidelines provided herein.

Metallic components having complex internal and external shapes may be produced using metal-injection-molding (“MIM”) processes. MIM and feedstocks for use therein have been described, for example, in U.S. Pat. Nos. 4,694,881, 4,694,882, 5,040,589, 5,064,463, 5,577,546, 5,848,350, 6,860,316, 6,838,046, 6,790,252, 6,669,898, 6,619,370, 6,478,842, 6,470,956, 6,350,328, 6,298,901, 5,993,507, 5,989,493, and U.S. patent application entitled “Metal Injection Molding of Spinal Fixation Systems Components,” bearing attorney docket number 64118.000190, filed concurrently herewith, the disclosures of each of which are incorporated herein in their entireties.

In general, the MIM process involves mixing a powder metal or metal alloy and a binder. Preferably, the mixture comprises a binder that is an organic aqueous based gel, and the mixture further comprises water. The mixed powder metal and binder composition preferably produces a generally flowable thixotropic mixture at relatively low temperature and pressure. The proportion of binder to powder metal may be about 40-60% binder by volume. Preferably, a flowable mixture with a viscosity is produced such that the mixture will fill all of the crevices and small dimensional features of a mold. The flowable mixture typically may be transferred to the mold via an injection molding machine.

Injection molding machines are known in the art and typically are capable of applying several hundred tons of pressure to a mold. The mold may be constructed with internal cooling passages to solidify the flowable material prior to removal. The mold cavity typically is larger than that of the desired finished part to account for the shrinkage that may occur after binder removal. The mold structure may be formed from either a rigid or a flexible material, such as metal, plastic, or rubber. Preferably, the mold is equipped with vents or bleeder lines to allow air to escape from the mold during the molding process. Alternatively, the mold may be equipped with a porous metal or ceramic insert to allow air to escape from the mold. After the mold has been filled with the flowable mixture, pressure may be applied to the mold/mixture to form the molded part, otherwise known as the preform. Typical injection mold pressures for a preform are in the range of about 10-12 ksi. The molded preforms may be referred to as “green” parts. The green preform may be dried by oven heating to a temperature sufficient to vaporize most of the remaining water. Then, the preform may be placed in a furnace to vaporize the binder. To achieve a part with high density and thus a sufficient working strength, the preform subsequently may be sintered.

Sintering is an elevated temperature process whereby a powder metal preform may be caused to coalesce into an essentially solid form having the same or nearly the same mechanical properties as the material in cast or wrought form. Generally, sintering refers to raising the temperature of the powder metal preform to a temperature close to, but not exceeding, the melting point of the material, and holding it there for a defined period of time. Under these conditions, interparticulate melting occurs and the material densifies to become solid.

In the case of MIM processes, the sintering process preferably causes interparticulate melting within the metallic component of the part while at the same time removing the binder component, which melts and vaporizes at a much lower temperature than does the metallic component. The resulting structure may be a high-density metallic piece substantially or completely free of the binder component. MIM molding facilitates the production of smaller and more dimensionally complex metallic pieces than does typical forging or casting processes because of the flexibility of the injection molding step in the process. One skilled in the art will appreciate the modifications of the basic MIM process that may be used in the embodiments, in accordance with the guidelines herein.

In another embodiment, PEEK as described herein may be substituted with different second material in the composite component(s) of the spinal fixation system. For example, the second material of the composite may be a nonresorbable polymer such as a member of the polyaryletherketone family (including polyetheretherketone), polyurethanes, silicone polyurethanes, polyimides, polyetherimides, polysulfones, polyethersulfones, polyaramids, polyphenylene sulfides, and any other non-resorbable polymer. In another embodiment, the second material of the composite may be a resorbable polymer such as polylactides (PLA), polyglycolide (PGA), copolymers of (PLA and PGA), polyorthoesters, tyrosine, polycarbonate, and any other resorbable or degradable polymer. The second material may be mixed or combined with a first material comprising a metal or metal alloy. A composite comprising the first material and the second material may be used to fabricate various components of a spinal fixation system, such as rods or plates, as has been described herein in regards to PEEK. The composites comprising a first material and second material as described herein may be advantageously used to fabricate spinal fixation system components having average or mean properties not otherwise attainable for a given dimension or size when using non-composite materials to fabricate the components.

The foregoing detailed description is provided to describe the invention in detail, and is not intended to limit the invention. Those skilled in the art will appreciate that various modifications may be made to the invention without departing significantly from the spirit and scope thereof.

What is claimed is:

1. A spinal fixation system comprising at least one composite component, the composite comprising:
a first material comprising at least one metal or metal alloy; and
a second material selected from the group consisting of resorbable and non-resorbable polymeric materials.

2. The system of claim 1, wherein the resorbable polymeric material is a material selected from the group consisting of polylactides (PLA), polyglycolide (PGA), copolymers of (PLA and PGA), polyorthoesters, tyrosine, polycarbonates, and mixtures and combinations thereof.

3. The system of claim 1, wherein the non-resorbable polymeric material is a material selected from the group consisting of members of the polyaryletherketone family, polyurethanes, silicone polyurethanes, polyimides, polyetherimides, polysulfones, polyethersulfones, polyaramids, polyphenylene sulfides, and mixtures and combinations thereof.

4. The system of claim 1, wherein the at least one metal or metal alloy is selected from the group consisting of titanium, titanium alloys, tantalum, tantalum alloys, stainless steel alloys, cobalt-based alloys, cobalt-chromium alloys, cobalt-chromium-molybdenum alloys, niobium alloys, zirconium alloys, and mixtures thereof.

5. The system of claim 1, wherein the composite component is a rod or plate.

6. A spinal fixation system, comprising at least one component comprised of a composite of polyetheretherketone (PEEK) and at least one metal or metal alloy.

7. The system of claim 6, wherein the component is a rod, and PEEK forms a core of the rod and the at least one metal or metal alloy forms a sheath around the core.

8. The system of claim 6, wherein the component is a rod, and the at least one metal or metal alloy forms a core of the rod and PEEK forms a sheath around the core.

9. The system of claim 6, wherein the component is a plate, and PEEK forms a core of the plate and the at least one metal or metal alloy forms a sheath around the core.

10. The system of claim 6, wherein the component is a plate, and the at least one metal or metal alloy forms a core of the plate and PEEK forms a sheath around the core.

11. The system of claim 6, wherein the component is a plate comprising at least one aperture.

12. The system of claim 6, wherein the at least one metal or metal alloy is selected from the group consisting of titanium, titanium alloys, tantalum, tantalum alloys, stainless steel alloys, cobalt-based alloys, cobalt-chromium alloys, cobalt-chromium-molybdenum alloys, niobium alloys, zirconium alloys, and mixtures thereof.

13. The system of claim 6, wherein the average or mean modulus of elasticity of the component is less than about 75 GPa.

14. The system of claim 6, wherein the average or mean tensile strength of the component is less than about 150 MPa.

15. A method of making a composite component of a spinal fixation system, comprising:
selecting a suitable design for the component; and
forming the composite component from at least one metal or metal alloy and a polymeric material selected from the group consisting of resorbable and non-resorbable polymeric materials.

16. The method of claim 15, wherein the resorbable polymeric material is a material selected from the group consisting of polylactides (PLA), polyglycolide (PGA), copolymers of (PLA and PGA), polyorthoesters, tyrosine, polycarbonates, and mixtures and combinations thereof.

17. The method of claim 15, wherein the non-resorbable polymeric material is a material selected from the group consisting of members of the polyaryletherketone family, polyurethanes, silicone polyurethanes, polyimides, polyetherimides, polysulfones, polyethersulfones, polyaramids, polyphenylene sulfides, and mixtures and combinations thereof.

18. The method of claim 15, wherein the at least one metal or metal alloy is selected from the group consisting of titanium, titanium alloys, tantalum, tantalum alloys, stainless steel alloys, cobalt-based alloys, cobalt-chromium alloys, cobalt-chromium-molybdenum alloys, niobium alloys, zirconium alloys, and mixtures thereof.

19. The method of claim 15, wherein the polymeric material is PEEK.

20. The method of claim 15, wherein forming the composite component comprises molding a core of a polymeric material and coating the core with the at least one metal or metal alloy.

21. The method of claim 15, wherein forming the composite component comprises molding a core of the at least one metal or metal alloy and coating the core with a polymeric material.

22. The method of claim 15, wherein forming the composite component comprises molding a core of a polymeric material and molding an outer sheath of at least one metal or metal alloy that is positioned around the core.

23. The method of claim 15, wherein forming the composite component comprises molding a core of at least one metal or metal alloy and molding an outer sheath of a polymeric material that is positioned around the core.

24. The method of claim 19, wherein the average or mean modulus of elasticity of the composite component is less than about 75 GPa.

25. The method of claim 19, wherein the average or mean tensile strength of the composite component is less than about 150 MPa.

26. The method of claim 15, wherein forming the composite component comprises metal injection molding the metal or metal alloy to include a cavity, and then filling the cavity with the polymeric material.
